(19) United States
(12) Patent Application Publication
(10) Pub. No.: US 2007/0272245 A1
(43) Pub. Date: Nov. 29, 2007

(54) SYSTEM AND METHOD FOR AN ANESTHESIA BREATHING CIRCUIT

(76) Inventors: Stephen Ripple, Glendale, AZ (US); Albert P. Martinez, Glendale, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/383,846

(22) Filed: May 17, 2006

(51) Int. Cl. A61M 16/00; A62B 7/10

(52) U.S. Cl. 128/205.28; 128/205.17

(57) ABSTRACT

A breathing bag, mask and/or tube of an anesthesia breathing circuit having printed thereon one or more of characters, stimuli, images, reflective surfaces, activity panels, puzzles, trivia, pleasing colors, decorative features and/or other calming and friendly items or indicia is disclosed. The enhancements may be visible by the patient prior, during and/or after the surgical procedure. The invention may also include any other item which enhances or supplements medical equipment such that the equipment is much more pleasing to a child during a surgical procedure.

### FIELD OF THE INVENTION

The present invention generally relates to an anesthesia breathing device, and more specifically, to calming images on any portion of an anesthesia breathing circuit such as, for example, the breathing bag, tubes and/or mask.

### BACKGROUND

An operating room typically includes large complex machines, needles, surgical tools, packets of blood and other serums, medicines and people behind masks, all in a very bland and sterile environment. Physicians often perform surgeries and other medical procedures in these operating rooms on a large number of children. Understandably, children are usually very afraid of the surgical procedures and do not like staying in a hospital, and particularly, do not like being in an operating room. While some hospitals paint the lobby walls more friendly colors and provide toys and play areas in the waiting areas, most operating rooms restrict child-friendly items. Some hospitals may also restrict children from bringing their favorite stuffed animal, toy, or other comfort item with them to the operating room, and even the parents of the child are often restricted from being in the surgical room with the child.

As is well known, a child's ability to see a friendly face, enjoyable character or pleasing pattern, often will relax a child and bring some joy to the child in an otherwise intimidating and unfriendly environment. Furthermore, it is important for the child to be calm at the start of a surgical procedure requiring anesthesia as this allows the anesthesiologist to supplement the child's breathing with pure oxygen via a mask. If a child is frightened and uncooperative, the mask is often forcibly held against the child's face until the blood oxygenation level is adequate. The more cooperative and calm the child, the safer it is to proceed with administering anesthesia. It is also very important for a child to relax before a surgical procedure because it may enhance the child's mental state and may improve the child's bodily functions before, during and after the procedure. A need exists to modify operating rooms by enhancing items within the operating room such that the items are much more pleasing to a child to facilitate the safety and comfort of administering anesthesia.

### SUMMARY OF THE INVENTION

The invention includes any item which enhances or supplements medical (e.g., anesthesia) equipment such that the equipment is much more pleasing to a child. The enhancements may be visible by the patient prior, during and/or after the surgical procedure. In one embodiment, the medical equipment is an anesthesia breathing circuit which includes a breathing bag, face mask and/or tubes and the item printed on the breathing circuit includes one or more of characters, stimuli, images, reflective surfaces, activity panels, puzzles, trivia, pleasing colors, decorative features and/or other calming and friendly items or indicia.

### DESCRIPTION OF EXEMPLARY DRAWINGS

Additional aspects of the present invention will become evident upon reviewing the non-limiting embodiments described in the specification and the claims, in conjunction with the accompanying figures, wherein like numerals designate like elements, and wherein:

FIG. 1 shows an anesthesia breathing circuit with an un-inflated bag in accordance with an exemplary embodiment of the present invention; and

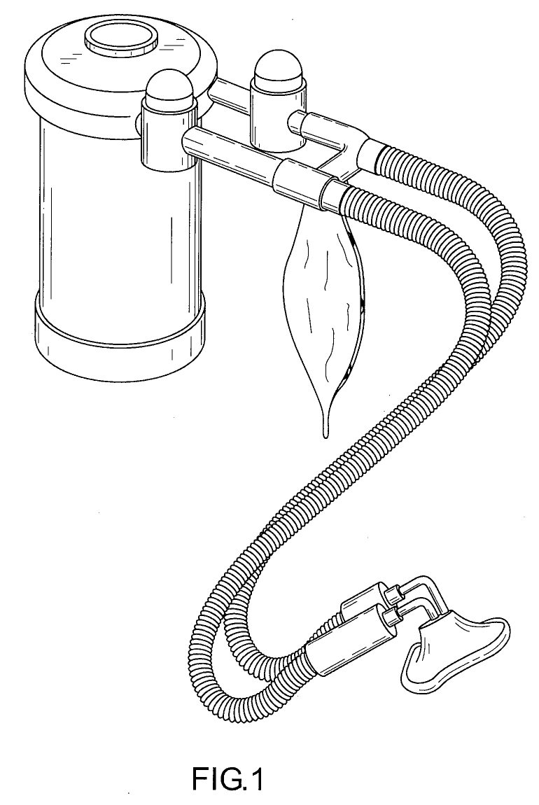

FIG. 2 shows an inflated breathing bag, face mask and tubes, which all show cartoon type characters, in accordance with an exemplary embodiment of the present invention.

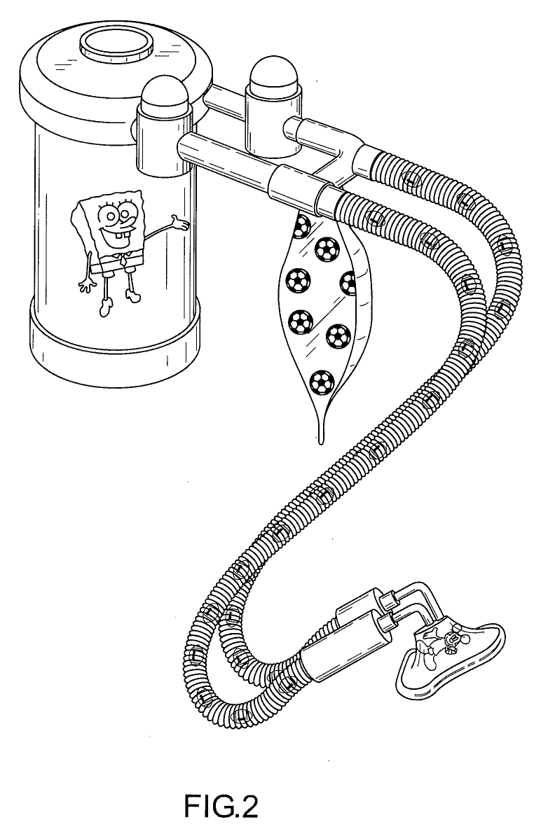

### DETAILED DESCRIPTION OF EXEMPLARY EMBODIMENTS

The following disclosure presents and describes various exemplary embodiments in sufficient detail to enable those skilled in the art to practice the invention, and it should be understood that other embodiments may be realized without departing from the spirit and scope of the invention. Thus, the following detailed description is presented for purposes of illustration only, and not of limitation, and the scope of the invention is defined solely by the appended claims.

The present invention includes any item which enhances or supplements medical equipment such that the equipment is much more pleasing to a child. The enhancements may be visually distinct from the surrounding surface and be visible by the patient prior, during and/or after the medical procedure. As used herein, an “item” includes any one or more of the enhancements or supplements disclosed herein and/or known to one skilled in the art now or in the future.

One of the devices often found in an operating room is an anesthesia breathing circuit. An anesthesia breathing circuit typically includes, for example, a breathing bag, a face mask, an anesthesia machine, a relief valve (pop-off valve), a device for removing carbon dioxide (absorber), conduits for carrying the gas to and from the patient (breathing tubes), and various valves to establish unidirectional flow for the gases. In one embodiment, the breathing bag, mask and/or tubes may include any one or more of the items discussed herein. The items may only be partially or fully viewable by the child when the breathing bag is inflated, thereby encouraging the child to breath into the bag in order to see the item. Items on other portions of the device may be viewable at all times, or during certain time periods or during certain actions (e.g., lights on, lights off, machine on, machine off, anesthesia at a certain level, etc).

In one embodiment, devices in operating rooms (e.g., breathing bags, masks and/or tubes) are enhanced by adding characters, stimuli, visual exhibits, light devices, audio devices, noise makers, images, reflective surfaces, holograms, activity panels, puzzles, trivia, pleasing colors, decorative features and/or other calming and friendly items or indicia to the devices. The characters and images may include, for example, cartoon characters (e.g., Spongbob, PowerRangers, Teletubbies), animals (e.g., dog, cat), sports items (e.g., baseball, football, soccer ball), sports figures (e.g., Michael Jordan's face, Steve Nash's face, Barry Bond's face), historical figures (e.g., Abraham Lincoln, Margaret Thatcher, Martin Luther King), historical landmarks (e.g., Grand Canyon, Eiffel Tower, Great Pyramid), movie or television stars (e.g., Brittney Spears, Clint Eastwood), and/or the like. The audio devices may include, for example, squeakers, announcements, or music makers which may be incorporated into the item, or which may be a complementary device. For example, the picture may include an elephant on the breathing bag and the operating room may include a recording of elephant sounds. In another example, every time the bag inflates, it activates a noise device which provides elephant sounds.

The items may be common items that are known to most children, or the items may include customized items which are specifically desired by a child. For example, the customized item may include a picture of a child's grandparent, the child's favorite scenery, and/or the child's favorite vacation spot which is printed on the device.

The items may also include interactive or “moving” images. For example, the item may include a clown face such that when the item moves, the clown face appears to smile. The item may also include a picture of a bear such that when the item moves, the bear appears to dance. The item may also change images, colors or other indicia upon certain occurrences. For example, the item may include a yellow background that changes to blue after hot air hits the surface. The item may also change from Batman to Superman upon movement of the item at different angles. The item may also display a one-dimensional, two-dimensional and/or three-dimensional image (which may be optionally viewed with 3D glasses).

The item may change colors or images upon changing environment. For example, upon shutting off or dimming the lights in the room, the item may glow in the dark. The item may include advertisements, motivational slogans or other words or logos which may also change. Toys, candy, or other prizes may be removably affixed or permanently affixed to the item.

The item may include combinations of items to encourage a particular activity or instruction. For example, the item may include a boy playing with blocks, then a boy sleeping to remind the child that the next step in the medical procedure is for the child to start sleeping.

While the invention is disclosed with respect to a child, one skilled in the art will appreciate that the invention may also be applicable for any age person (e.g., child, teenager, adult, senior citizen), or animal. For example, the item may include a dog bone for a dog that needs to undergo surgery in a veterinarian's office.

The item may be created on any portion of the device by any method known in the art (e.g., screen printing, sewing, Velcro, heat sealing, etc). The item may also be permanently, temporarily and/or removably affixed to the device. For example, any portion of the item may be printed permanently on the device, or any portion of the item may be a sticker on the device which can be easily removed and replaced. A portion of the item may appear on one part of the device, while the other portion of the item may appear on a second portion of the device. For example, the upper portion of batman may appear on the breathing bag, while the lower portion of batman may appear on the mechanical equipment. The item may also be reversible. For example, the bag may be reversible such that one side includes a first item (e.g., Superman image), and the other side includes a second item (e.g., Batman image). The item may also be affixed to the inside of the device, or placed in the device (e.g., a clear bag). For example, a toy rabbit may be placed inside a clear bag, such that the rabbit bounces around during the breathing cycle.

In the foregoing specification, the invention has been described with reference to specific embodiments. However, it will be appreciated that various modifications and changes can be made without departing from the scope of the present invention as set forth in the claims below. The specification and figures are to be regarded in an illustrative manner, rather than a restrictive one, and all such modifications are intended to be included within the scope of present invention. Accordingly, the scope of the invention should be determined by the appended claims and their legal equivalents, rather than by the examples given above. For example, the steps recited in any of the method or process claims may be executed in any order and are not limited to the order presented in the claims.

Benefits, other advantages, and solutions to problems have been described above with regard to specific embodiments. However, the benefits, advantages, solutions to problems, and any element(s) that may cause any benefit, advantage, or solution to occur or become more pronounced are not to be construed as critical, required, or essential features or elements of any or all the claims. As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

We claim:

1. An anesthesia breathing circuit comprising:
at least one of a breathing bag, mask and tube having at least a portion of an item affixed thereto, wherein said item is calming to a patient in a surgical room;
an anesthesia machine;
a device configured to remove carbon dioxide;
conduits configured to carry gas to and from said patient; and,
a plurality of valves configured to establish unidirectional flow for said gases.

2. The circuit of claim 1, wherein said item is at least one of permanently, removably and temporarily affixed to said breathing bag.

3. The circuit of claim 1, wherein said patient comprises at least one of a person and animal.

4. The circuit of claim 1, wherein said item includes at least one of a character, stimuli, visual exhibit, light device, audio device, noise maker, image, reflective surface, hologram, activity panel, puzzle, trivia, and decorative feature.

5. The circuit of claim 1, further comprising at least one of a light device and noise device which correlates to a theme of said item.

6. The circuit of claim 1, wherein said item is located inside said breathing bag.

7. The circuit of claim 1, wherein said item is affixed to an inside surface of said breathing bag.
